(19) United States
(12) Patent Application Publication
(10) Pub. No.: US 2023/0144869 A1
(43) Pub. Date: May 11, 2023

(54) METHODS FOR TREATING CYTOKINE RELEASE SYNDROME

(71) Applicant: UNIVERSITY HEALTH NETWORK, Toronto (CA)

(72) Inventors: Mark R. Bray, Oakville (CA); Jacqueline M. Mason, Toronto (CA); Xin Wei, Markham (CA); Gordon Duncan, Toronto (CA)

(21) Appl. No.: 17/917,995

(22) PCT Filed: Apr. 12, 2021
(86) PCT No.: PCT/CA2021/050483

§ 371 Date: Oct. 10, 2022

Related U.S. Application Data
(60) Provisional application No. 63/022,956, filed on May 11, 2020.
(60) Provisional application No. 63/009,059, filed on Apr. 13, 2020.

(51) Int. Cl. A61K 31/496; A61P 37/06

(52) U.S. Cl. CPC A61K 31/496; A61P 37/06

(57) ABSTRACT

Disclosed herein is a method of treating a subject with aberrant cytokine release from a disease or condition or at risk of developing aberrant cytokine release from a disease or condition. The method comprises administering to the subject an effective amount of a compound represented by structural formula (I): (I) or a pharmaceutically acceptable salt thereof. The variables in structural formula (I) are as described herein.

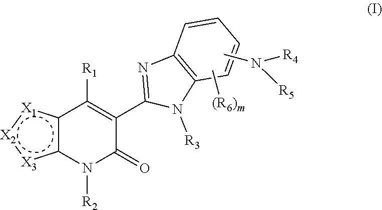

### CROSS-REFERENCE TO RELATED APPLICATIONS

This application claims priority to U.S. Provisional Application No. 63/009,059, filed Apr. 13, 2020, and U.S. Provisional Application No. 63/022,956, filed May 11, 2020. The entire contents of the aforementioned application are incorporated herein by reference.

### BACKGROUND

Cytokine release syndrome is a systemic inflammatory response that can be triggered by a variety of factors such as infections and certain drugs. Severe cases have been referred to as “cytokine storm syndrome”. Symptoms include fever, fatigue, loss of appetite, muscle and joint pain, nausea, vomiting, diarrhea, rashes, fast breathing, rapid heartbeat, low blood pressure, seizures, headache, confusion, delirium, hallucinations, tremor and loss of coordination. Lab tests and clinical monitoring show low blood oxygen, widened pulse pressure, increased cardiac output (early), potentially diminished cardiac output (late), high levels of nitrogen compounds in the blood, elevated D-dimer, elevated transaminases, factor I deficiency and excessive bleeding and higher-than-normal level of bilirubin.

Cytokine release syndrome occurs when large numbers of white blood cells are activated and release inflammatory cytokines, which in turn activate yet more white blood cells in a positive feedback loop of pathogenic inflammation. This can occur when the immune system is fighting pathogens, as cytokines produced by immune cells recruit more effector immune cells such as T-cells and inflammatory monocytes (which differentiate into macrophages) to the site of inflammation or infection. In addition, pro-inflammatory cytokines binding their cognate receptor on immune cells results in activation and stimulation of further cytokine production. This process, when dysregulated, can be life-threatening due to systemic hyper-inflammation, hypotensive shock, and multi-organ failure.

The term “cytokine release syndrome” was first coined in the early '90s, when the anti-T-cell antibody muromonab-CD3 (OKT3) was introduced into the clinic as an immunosuppressive treatment for solid organ transplantation [Chatenoud L, et al., N Engl J Med. 1989; 320:1420-1421; Chatenoud L, et al., Transplantation. 1990; 49:697-702]. Subsequently, Cytokine Release Syndrome has been described after infusion of several antibody-based therapies, such as anti-thymocyte globulin (ATG) [Pihusch R, et al., Bone Marrow Transplant. 2002; 30:347-354], the CD28 superagonist TGN1412 [Suntharalingam G, et al., N Engl J Med. 2006; 355:1018-1028], rituximab [Winkler U, et al., Blood. 1999; 94], obinutuzumab [Freeman C L, et al., Blood. 2015; 126], alemtuzumab [Wing M G, et al., J Clin Invest. 1996; 98:2819-2826], brentuximab [Alig S K, et al., Eur J Haematol. 2015; 94:554-557], dacetuzumab [de Vos S, et al., J Hematol Oncol. 2014; 7:44], and nivolumab [1Rotz S J, et al., Pediatr Blood Cancer. 2017; 64:e26642]. Cytokine Release Syndrome has also been observed following administration of non-protein-based cancer drugs, such as oxaliplatin [Tonini G, et al., J Biol Regul Homeost Agents. 2002; 16:105-109] and lenalidomide [Aue G, et al., Haematologica. 2009; 94:1266-1273]. Furthermore, Cytokine Release Syndrome was reported in the setting of haploidentical donor stem cell transplantation, and graft-versus-host disease (GVHD) [Abboud R, et al., Biol Blood Marrow Transplant. 2016; 22:1851-1860, Cho C, et al., Bone Marrow Transplant. 2016; 51:1620-1621]. Cytokine storm due to massive T-cell stimulation is also a proposed pathomechanism of viral infections, such as influenza [Tisoncik J R, et al., Microbiol Mol Biol Rev. 2012; 76:16-32, de Jong M D, et al., Nat Med. 2006; 12:1203-1207].

Lately, with the success of the newer T-cell-engaging immunotherapeutic agents there has been a growing interest in Cytokine Release Syndrome since it represents one of the most frequent serious adverse effects of these therapies. For example, studies with blinatumomab [Teachey D T, et al., Blood. 2013; 121:5154-5157] and CD19-targeted CAR T cells [Morgan R A, et al., ERBB2. Mol Ther. 2010; 18:843-851; Brudno J N, Kochenderfer J N. Blood. 2016; 127(26):3321-30; and Porter D L, et al., N Engl J Med. 2011; 365:725-733] revealed that Cytokine Release Syndrome is the most important adverse event of these therapies with frequencies of up to 100% in CD19-targeted CAR T cell trials, sometimes with fatal outcome.

Cytokine Release Syndrome is also associated with coronavirus disease 2019 (COVID-19). As of Apr. 12, 2020, coronavirus disease 2019 has been confirmed in 1,696,588 people worldwide, carrying a mortality of approximately 6.2% (Coronavirus disease 2019 (COVID-19) situation report—52. Apr. 12, 2020). Accumulating evidence suggests that a subgroup of patients with severe COVID-19 develop Cytokine Storm Syndrome, which contributes to the high rate of mortality in this subgroup of patients.

There is therefore an urgent need for developing effective therapies because of inter alia the health emergency cause by the coronavirus and influenza virus and with the increased use of T-cell-engaging immunotherapeutic agents.

### SUMMARY

It has now been found that the compounds depicted herein inhibit the aberrant release of cytokines. For example, Compound 1 suppresses human immune cell activation, proliferation and cytokine production in in vitro assays that simulate certain aspects of cytokine release syndrome. For example, Compound 1 treatment of peripheral blood mononuclear cells inhibits CD4+ and CD8⁺ T-cell activation and proliferation induced by several stimuli, including anti-CD3 and anti-CD28 antibodies, phytohemagglutinin and the superantigen staphylococcal enterotoxin B (Example 1); Compound 1 treatment of peripheral blood mononuclear cells inhibits lymphocyte proliferation in an allogenic mixed lymphocyte reaction (Example 2); Compound 1 treatment of peripheral blood mononuclear cells suppresses anti-CD3 antibody and anti-CD28 antibody-stimulated release of cytokines, including IL-2, IL-6, IFNγ and TNFα (Example 3); Compound 1 inhibits TGFβ cytokine production by mouse primary cancer-associated fibroblasts (example 3); Compound 1 treatment promotes loss of cell viability in resting CD14′ monocytes (Example 4); and Compound 1 does not cause cytokine production in unstimulated whole blood, and therefore is not expected to cause cytokine release syndrome in patients (Example 6). In addition, Compound 2 blocks disease progression in an animal model of multiple sclerosis [i.e., experimental autoimmune encephalomyelitis (EAE)] (Example 6). Based in part on these results, methods of inhibiting aberrant cytokine release and systemic inflammation in subjects are disclosed herein.

The invention is a method of treating a subject with aberrant cytokine release from a disease or condition or at risk of developing aberrant cytokine release from a disease or condition. The method comprises administering to the subject an effective amount of a compound represented by structural formula (I):

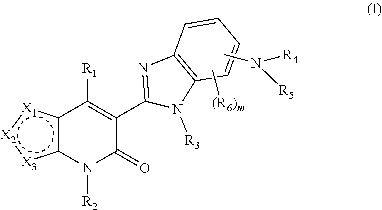

or a pharmaceutically acceptable salt thereof, wherein:

one of X₁, X₂, and X₃ is S, the other two are each independently CR;

R is H, —F, —Cl, —Br, —OH, —(C₁-C₄)alkyl, —(C₁-C₄)haloalkyl, —(C₁-C₄)alkoxy, —(C₁-C₄)alkylene-OH or 4-7 membered monocyclic heterocyclyl optionally substituted with 1-3 groups selected from —F, —Cl, —Br, —OH, —(C₁-C₄)alkyl, —(C₁-C₄)haloalkyl, —(C₁-C₄)alkoxy, or —CO₂—(C₁-C₄)alkyl;

R₁ is —NR^{a}R^{b} or —OR^{a1}; R^{a} for each occurrence is independently —H, —(C₁-C₆)alkyl, —(CH₂)ₙ—(C₃-C₇)cycloalkyl, —(CH₂)ₙ-3-7 membered monocyclic heterocyclyl, —(CH₂)ₙ-bridged (C₆-C₁₂)cycloalkyl, optionally substituted —(CH₂)ₙ-5-10 membered heteroaryl; or —(CH₂)ₙ-6-12 membered bridged heterocyclyl, wherein —(C₁-C₆)alkyl, —(CH₂)ₙ—(C₃-C₇)cycloalkyl, —(CH₂)ₙ-3-7 membered monocyclic heterocyclyl, —(CH₂)ₙ-bridged (C₆-C₁₂)cycloalkyl, —(CH₂)ₙ-5-10 membered heteroaryl, or —(CH₂)ₙ-6-12 membered bridged heterocyclyl, is optionally substituted with 1-3 groups selected from —F, —Cl, —Br, —CN, —NH₂, —OH, oxo, —(C₁-C₄)alkyl, —(C₁-C₄)haloalkyl, —(C₁-C₄)alkoxy, —(C₁-C₄)haloalkoxy, —(C₁-C₄)alkylene-OH, or —(C₁-C₄)alkylene-NH₂;

R^{b} for each occurrence is independently —H or —(C₁-C₆)alkyl; or,

R^{a} and R^{b}, together with the nitrogen to which they are attached, form —(C₃-C₁₀)heterocyclyl;

R^{a1} for each occurrence is independently —H, (C₁-C₆)alkyl, (C₃-C₁₀)cycloalkyl, 3-10 membered heterocyclyl, (C₆-C₁₀)aryl, or 3-10 membered heteroaryl;

R₂ and R₃ are independently H or —(C₁-C₄)alkyl;

R₄ and R₅, together with the nitrogen to which they are attached, form 4-7 membered monocyclic heterocyclyl or 6-12 membered bridged heterocyclyl, wherein the 4-7 membered monocyclic heterocyclyl or 6-12 membered bridged heterocyclyl is optionally substituted with 1-3 groups selected from —F, —Cl, —Br, —CN, —NH₂, —OH, oxo, —(C₁-C₄)alkyl, —(C₁-C₄)haloalkyl, —(C₁-C₄)alkoxy, —(C₁-C₄)haloalkoxy, —(C₁-C₄)alkylene-OH, or —(C₁-C₄)alkylene-NH₂;

R₆ for each occurrence is independently —F, —Cl, —Br, —CN, —NH₂, —OH, —(C₁-C₆)alkyl, —(C₁-C₆)haloalkyl, —(C₂-C₆)alkenyl, —(C₂-C₆)alkynyl, (C₃-C₆)cycloalkyl, —(C₁-C₆)alkoxy, —(C₁-C₆)haloalkoxy, —(C₁-C₆)alkylene-OH, or —(C₁-C₆)alkylene-NH₂;

m is 0, 1, 2, or 3; and

n is 0, 1, or 2.

Another embodiment of the invention is a method of treating a subject with a systemic inflammatory response from a disease or condition or a subject at risk of developing systemic inflammatory response from a disease or condition, comprising administering to the subject a compound of structural formula (I), or a pharmaceutically acceptable salt thereof.

Another embodiment of the invention is a compound disclosed herein (e.g., a compound of structural formula (I), or a pharmaceutically acceptable salt thereof) for treating a subject with aberrant cytokine release from a disease or condition or at risk of developing aberrant cytokine release from a disease or condition.

Another embodiment of the invention is a compound disclosed herein (e.g., a compound of structural formula (I), or a pharmaceutically acceptable salt thereof) for treating a subject with a systemic inflammatory response from a disease or condition or a subject at risk of developing systemic inflammatory response from a disease or condition.

Also disclosed is the use of a compound disclosed herein (e.g., a compound of structural formula (I), or a pharmaceutically acceptable salt thereof) for the manufacture of a medicament for treating a subject with aberrant cytokine release from a disease or condition or at risk of developing aberrant cytokine release from a disease or condition.

Also disclosed is the use of a compound disclosed herein (e.g., a compound of structural formula (I), or a pharmaceutically acceptable salt thereof) for the manufacture of a medicament for treating a subject with a systemic inflammatory response from a disease or condition or a subject at risk of developing systemic inflammatory response from a disease or condition

### BRIEF DESCRIPTION OF DRAWINGS

FIG. TA shows that the Compound 1 treatment of peripheral blood mononuclear cells (PBMCs) resulted in a titratable inhibition of CD4⁺ and CD8⁺ T-cell activation by anti-CD3 and anti-CD28 antibodies, phytohemagglutinin (PHA) or staphylococcal enterotoxin B (SEB) as shown by reduced cell surface expression of CD25 (IL-2 receptor alpha chain) and CD69 (type II C-lectin receptor), and reduced shedding of CD62L (L-selectin). FIG. 1B shows proliferation of anti-CD3 antibody and anti-CD28 antibody-, PHA- or SEB-activated lymphocytes was inhibited by Compound 1 treatment.

FIG. 2A, FIG. 2B and FIG. 2C show that Compound 1 inhibits the proliferation of lymphocytes in an allogenic mixed lymphocyte reaction (MLR) in a dose-dependent manner.

FIG. 3A shows that the level of all measured cytokines, including IL-2, IL-6, IFNγ and TNFα, decreased in anti-CD3 antibody and anti-CD28 antibody-activated PBMCs in the presence of Compound 1. FIG. 3B shows that Compound 1 inhibited TGFβ cytokine production by mouse primary cancer-associated fibroblasts (CAFs).

FIG. 4 shows that Compound 1 treatment led to a dose-dependent loss of cell viability in resting CD14⁺ monocytes, but had no significant effect on the cell viability of resting CD4⁺ and CD8⁺ T cells except at high concentrations (30 μM).

FIG. 5 shows that Compound 2 blocks experimental autoimmune encephalomyelitis (EAE) disease progression in mice.

### DETAILED DESCRIPTION

The invention is directed towards treating a subject with aberrant cytokine release from a disease or condition. The invention is also directed towards treating a subject at risk of developing aberrant cytokine release from a disease or condition. There are many diseases and conditions which involve an inflammatory and/or immune response, which are mediated by cytokine release. An inflammatory and/or autoimmune response is a healthy and desirable defense mechanism to, for example, infection by a pathogen, where an inflammatory response by the immune system is intended to eradicate the pathogen. When pathogen has been eradicated, the immune response recedes and the patient recovers.

In some instances, a subject experiences an aberrant release of cytokines during an immune response, i.e., a cytokine release that is too long in duration, resulting in a chronic inflammatory condition, or too strong in magnitude, resulting in an acute inflammatory condition. The consequence of aberrant cytokine release is an immune system that is out control. Such is believed to be the case, for example, in the subgroup of COVID-19 patients who experience severe symptoms; in attempting to respond to the viral infection, the immune system over responds, leading to severe illness and even death. Another example is the excessive immune response that sometimes occurs with chimeric antigen receptor (CAR) T cell therapy, i.e., a severe and potentially life threatening condition resulting from a massive release of cytokines. These aberrant releases of cytokines, particularly when resulting in symptoms characterized by hyperinflammation, are often referred to as “cytokine release syndrome”. The invention is therefore directed towards treating subjects with hyperinflammation or systemic inflammation from a disease or condition or who are at risk of developing hyperinflammation or systemic inflammation from a disease or condition. “Cytokine release syndrome” refers to a systemic inflammatory response resulting from the inappropriate positive signaling between cytokines and immune cells and ultimately to excessive levels of cytokine release. It occurs when large numbers of white blood cells are activated and release inflammatory cytokines, which in turn activate yet more white blood cells in a positive feedback loop of pathogenic inflammation. The cytokines produced by immune cells recruit more effector immune cells such as T-cells and inflammatory monocytes (which differentiate into macrophages) to the site of inflammation or infection. In addition, pro-inflammatory cytokines binding their cognate receptor on immune cells results in activation and stimulation of further cytokine production. In patients this leads to a high fever, swelling and redness, extreme fatigue, nausea and in some instances is fatal. More than 150 known inflammatory mediators are thought to be released during cytokine release syndrome, including IL-Iβ, TNFα, IL-6, IL-8 (CXCL8), IL-2, IL-10, IFNγ, IL-12p70 and GM-CSF.

“Cytokine storm syndrome” refers to severe cases of cytokine release syndrome.

Treating a “subject who is at risk of developing aberrant cytokine release from a disease or condition” means treating of patients with the disease or condition among whom it is known that a subgroup typically develops aberrant cytokine release (or cytokine release syndrome or cytokine storm syndrome). In some instances, it may be possible to identify individuals in the subgroup who are at risk and treat those at risk subjects only. In other instances, it may not be possible or practical to identify the subjects in the subgroup who are at risk, in which case subjects among the entire groups are treated, i.e., the invention contemplates treating some subjects who may never have experienced the aberrant release of cytokines. Subjects at risk of developing aberrant cytokine release from the disease or condition are preferably treated before the aberrant cytokine release occurs, e.g., before the onset of symptoms from the aberrant cytokine release occurs, to reduce the severity of symptoms, when they develop, or to delay the onset of the symptoms.

Conditions characterized by aberrant cytokine release and which can be treated by the disclosed methods include conditions resulting from therapies with activated T-cells, therapies with activated natural killer (NK) cells, therapies with activated dendritic cells, therapies with activated macrophages, therapies with activated B-cells, and antitumor cell therapy. Other conditions characterized by aberrant cytokine release and which can be treated by the disclosed methods include conditions resulting from adoptive cell therapy using tumor-infiltrating lymphocyte (TIL) therapy, engineered T cell receptor (TCR) therapy, chimeric antigen receptor (CAR) T cell therapy and therapies that incorporate other immune cells, such as NK cells. In one embodiment, the condition results from CAR T cell therapy, e.g., with tisagenlecleucel or axicabtagene ciloleucel. Subjects with these conditions can be treated according to the disclosed methods after the onset of symptoms and/or aberrant cytokine release. Alternatively, subjects with these conditions who are at risk of aberrant cytokine release can be treated before the onset of symptoms and/or before aberrant cytokine release.

CAR T Therapy involves T-cells that have been genetically engineered to produce an artificial T-cell receptor for use in immunotherapy. The artificial receptors are receptor proteins that have been engineered to combine both antigen-binding and T-cell activating functions into a single receptor. The T-cells are harvested either from the patient or a healthy donor, genetically altered to express a specific CAR and then infused. As such, they are programed to target an antigen that is present on the surface of tumors and not expressed on healthy cells. After CAR T cells are infused into a patient, CAR T cells bind to their targeted cell, become activated, then proceed to proliferate and become cytotoxic. CAR T cells destroy cells through several mechanisms, including extensive stimulated cell proliferation, increasing the degree to which they are toxic to other living cells (cytotoxicity) and by causing the increased secretion of factors that can affect other cells, such as cytokines, interleukins and growth factors.

Other conditions characterized by aberrant cytokine release and which can be treated by the disclosed methods include conditions resulting from therapies with antibodies. The antibody can be a monoclonal antibody, an antibody fragment, an Fc-fusion protein or a bispecific antibody (e.g., bispecific T cell engager or BiTE). Subjects with these conditions can be treated according to the disclosed methods after the onset of symptoms and/or aberrant cytokine release. Alternatively, subjects with these conditions who are at risk of aberrant cytokine release can be treated before the onset of symptoms and/or before aberrant cytokine release.

In a specific embodiment, conditions characterized by aberrant cytokine release and which can be treated by the disclosed methods include conditions resulting from therapies with a monoclonal antibody, including anti-PD-L1 antibody, an anti-CTLA-4 antibody, an anti-PD-1 antibody, anti-CD3 antibody, anti-CD20 antibody, anti-CD28 antibody, anti-CD52 antibody and anti-thymocyte globulin (ATG). Specific examples include Nivolumab, Muromonab, Rituximab, Brentuximab, Theralizumab, Alemtuzumab, Obinutuzumab, Dacetuzumab, Pembrolizumab, Cemiplimab, Atezolizumab, Avelumab, Durvalumab and Ipilimumab. In a specific embodiment, conditions characterized by aberrant cytokine release and which can be treated by the disclosed methods include conditions resulting from therapies with a bispecific T cell engager, including Blinatumomab (Blincyto). Subjects with these conditions can be treated according to the disclosed methods after the onset of symptoms and/or aberrant cytokine release. Alternatively, subjects with these conditions who are at risk of aberrant cytokine release can be treated before the onset of symptoms and/or before aberrant cytokine release.

Other conditions characterized by aberrant cytokine release and which can be treated by the disclosed methods include conditions resulting from therapies with a non-protein based cancer drugs, such as oxaliplatin and lenalidomide. Subjects with these conditions can be treated according to the disclosed methods after the onset of symptoms and/or aberrant cytokine release. Alternatively, subjects with these conditions who are at risk of aberrant cytokine release can be treated before the onset of symptoms and/or before aberrant cytokine release.

Other conditions characterized by aberrant cytokine release and which can be treated by the disclosed methods include conditions resulting from a haploidentical donor stem cell transplantation. Subjects with these conditions can be treated according to the disclosed methods after the onset of symptoms and/or aberrant cytokine release. Alternatively, subjects with these conditions who are at risk of aberrant cytokine release can be treated before the onset of symptoms and/or before aberrant cytokine release.

Diseases characterized by aberrant cytokine release and which can be treated by the disclosed methods include infectious diseases. The infectious disease can be viral, bacterial, fungal, helminthic, protozoan, or hemorrhagic. In one specific embodiment, the infection is a viral disease selected from influenza, Arenaviridae, Filoviridae, Bunyaviridae, Flaviviridae, Rhabdoviridae and Cornaviridae. Alternatively, the infection is a viral disease selected from Epstein Barr virus, small pox, Ebola, Marburg, Crimean-Congo hemorrhagic fever (CCHF), South American hemorrhagic fever, dengue, yellow fever, Rift Valley fever, Omsk hemorrhagic fever virus, Kyasanur Forest, Junin, Machupo, Sabia, Guanarito, Garissa, Ilesha and Lassa.

A small subgroup of subjects with Cornaviridae or influenza virus infections experience severe symptoms characterized by hyperinflammation, i.e., cytokine storm syndrome, which can lead to respiratory failure and even death. Included are Cornaviridae virus infection from SARS, SARS-CoV-2, MERS, 229E, NL63, OC43, and HKU1. Subjects with these viral infections can be treated according to the disclosed methods after the onset of symptoms and/or aberrant cytokine release. Alternatively, subjects with these viral diseases are at risk of aberrant cytokine release can be treated before the onset of symptoms and/or before aberrant cytokine release. In a specific embodiment, subjects who are particularly at risk of developing aberrant cytokine release are those having underlying conditions, for example, diabetes, cardiovascular disease (e.g., hypertension), chronic lung disease (e.g., severe asthma, chronic obstructive pulmonary disease or emphysema), age over 65, body mass index of 40 or higher, immunosuppression, chronic kidney disease, liver disease and lung damage due to smoking. Subjects particularly at risk have an HScore greater than 150, 160, 170 or 180. HScore is obtained by scoring key indicators of the likelihood of a subject developing aberrant cytokine release and summing each score to obtain a composite score that is predictive of developing aberrant cytokine release. See Fardet L, et al.,. Arthritis Rheumatol 2014; 66: 2613-20; and http://saintantoine.aphp.fr/score/ for an HScore calculator. IL-6 levels in the subject 2, 2.5, 2.75, 3.0 or 3.5 higher than normal are also predictive of a subject at higher risk of developing aberrant cytokine release.

Diseases characterized by aberrant cytokine release and which can be treated by the disclosed methods include auto-inflammatory diseases or an autoimmune diseases. Examples include Type 1 diabetes, Type 2 diabetes, rheumatoid arthritis (RA), systemic lupus erythematosus (SLE), multiple sclerosis (MS), inflammatory bowel disease (Crohn's disease and ulcerative colitis), psoriasis, asthma, familial Mediterranean fever (FMF), Tumor Necrosis Factor (TNF) receptor-associated periodic syndrome (TRAPS), mevalonate kinase deficiency/hyperimmunoglobulin D syndrome (MKD/HIDS), Muckle-Wells syndrome (MWS), familial cold autoinflammatory syndrome (FCAS), neonatal-onset multisystem inflammatory disease (NOMID), periodic fever, aphthous stomatitis, pharyngitis and adenitis (PFAPA syndrome), pyogenic sterile arthritis, pyoderma gangrenosum, acne (PAPA), deficiency of the interleukin-1 receptor antagonist (DIRA), Behcet's disease, Majeed Syndrome, Chronic recurrent multifocal osteomyelitis (CRMO), Schnitzler syndrome and Blau syndrome. Other examples include hemophagocytic lymphohistiocytosis (HLH), familial (primary) hemophagocytic lymphohistiocytosis (FHL), sporadic HLH, macrophage activation syndrome (MAS), chronic arthritis, systemic Juvenile Idiopathic Arthritis (sJIA), Still's Disease, a Cryopyrin-associated Periodic Syndrome (CAPS), Familial Cold Auto-inflammatory Syndrome (FCAS), Familial Cold Urticaria (FCU), Muckle-Well Syndrome (MWS), Chronic Infantile Neurological Cutaneous and Articular (CINCA) Syndrome, a cryopyrinopathy comprising inherited or de novo gain of function mutations in the NLRP3 gene, a hereditary auto-inflammatory disorder, acute pancreatitis, severe burn injury, acute radiation syndrome, trauma, acute respiratory distress syndrome, systemic inflammatory response syndrome, and tumor lysis syndrome. Other examples include cachexia, a chronic inflammatory response, sepsis, septic shock syndrome, traumatic brain injury, cerebral cytokine storm, graft-versus-host disease (GVHD), autoimmune diseases, multiple sclerosis (MS), acute pancreatitis, or hepatitis. Yet other examples include myocarditis, Type I diabetes, Type 2 diabetes, thyroiditis, uveitis, encephalomyelitis, arthritis (e.g., rheumatoid), lupus erythematosus, myositis, systemic sclerosis, Sjogren's syndrome and heart failure. Subjects with these conditions can be treated according to the disclosed methods after the onset of symptoms and/or aberrant cytokine release, or in subjects at risk of aberrant cytokine release.

Alternatively, the compound used in the disclosed methods is represented by Structural Formula (II-A), (II-B) or (II-C):

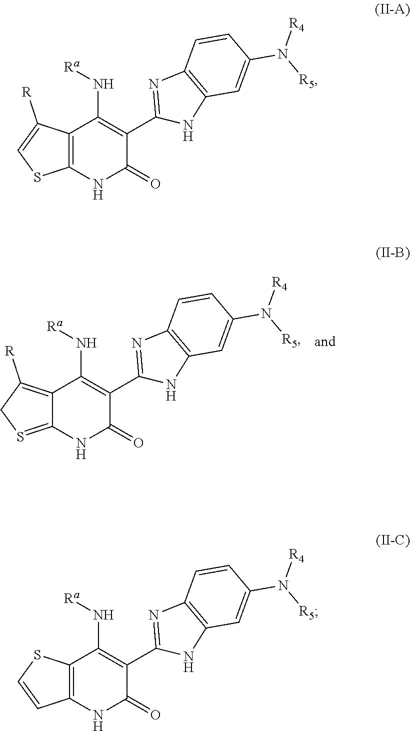

or a pharmaceutically acceptable salt thereof. The variables in Structural Formula (II-A), (II-B) or (II-C) are as described for Structural Formula (I).

In a first embodiment, the compound used in the disclosed methods represented by Structural Formula (II-A), (II-B) or (II-C) or a pharmaceutically acceptable salt thereof, wherein R is H, —(C₁-C₄)alkyl, —(C₁-C₄)alkoxy, N-piperazinyl optionally substituted with —CO₂—(C₁-C₄)alkyl; R₄ and R₅, together with the nitrogen to which they are attached, form —N-alkyl-piperazinyl or morpholinyl, wherein the piperazinyl or morpholinyl is optionally substituted with 1-2 groups selected from —F, —Cl, —Br, —OH, —(C₁-C₄)alkyl, —(C₁-C₄)haloalkyl, or —(C₁-C₄)alkoxy; and R^{a} for each occurrence is independently —H, —(CH₂)ₙ—(C₃-C₆)cycloalkyl, —(CH₂)ₙ-3-6 membered monocyclic heterocyclyl, wherein the —(CH₂)ₙ—(C₃-C₆)cycloalkyl or —(CH₂)ₙ-3-6 membered monocyclic heterocyclyl is optionally substituted with 1-3 groups selected from —F, —Cl, —Br, —CN, —NH₂, —OH, —(C₁-C₄)alkyl, or —(C₁-C₄)alkoxy; and n is 0 or 1.

In a second embodiment, the compound used in the disclosed methods is represented by Structural Formula (II-A), (II-B) or (II-C) or a pharmaceutically acceptable salt thereof, wherein R is H; R₄ and R₅, together with the nitrogen to which they are attached, form —N-methyl-piperazinyl or morpholinyl, both of which are optionally substituted with one or two methyl; R^{a} for each occurrence is independently —H; —(C₃-C₆)cycloalkyl optionally substituted with —OH; —(CH₂)ₙ-tetrahydro-2H-pyran; morpholinyl; piperidinyl optionally substituted with —F, —OH or methyl; or tetrahydrofuran; and n is 0 or 1.

The compounds depicted below and pharmaceutically acceptable salts thereof can also be used in the disclosed methods. The compounds used in the disclosed methods can be prepared according to procedures disclosed in WO2016/205942, the entire teachings of which are incorporated herein by reference.

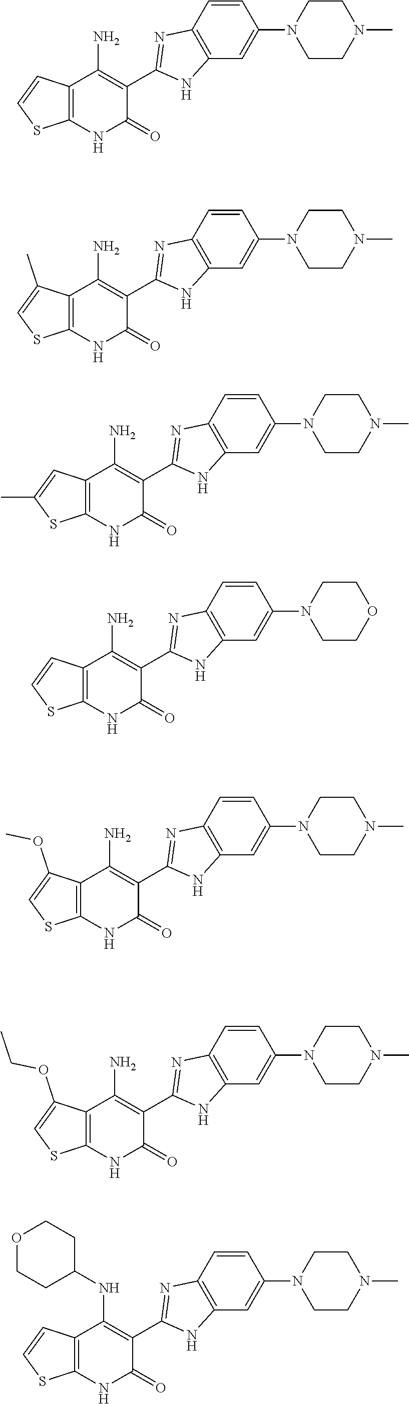

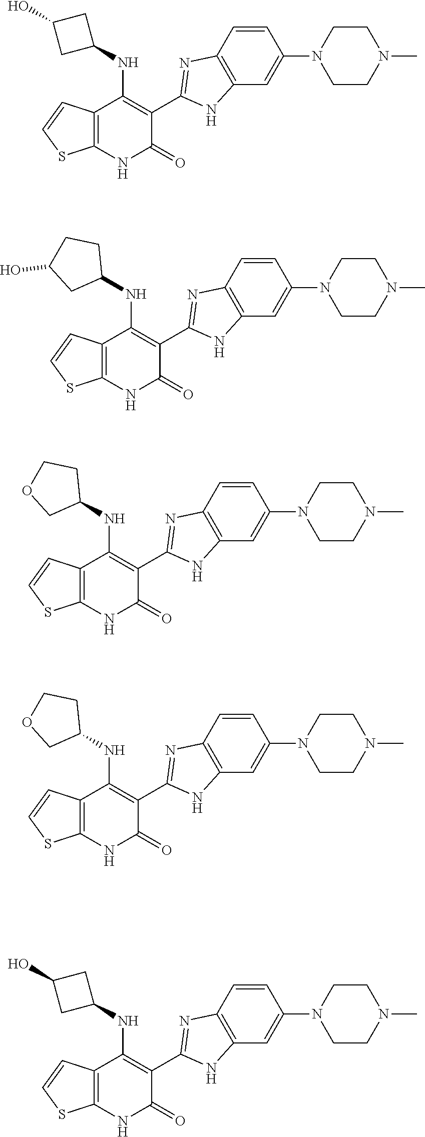

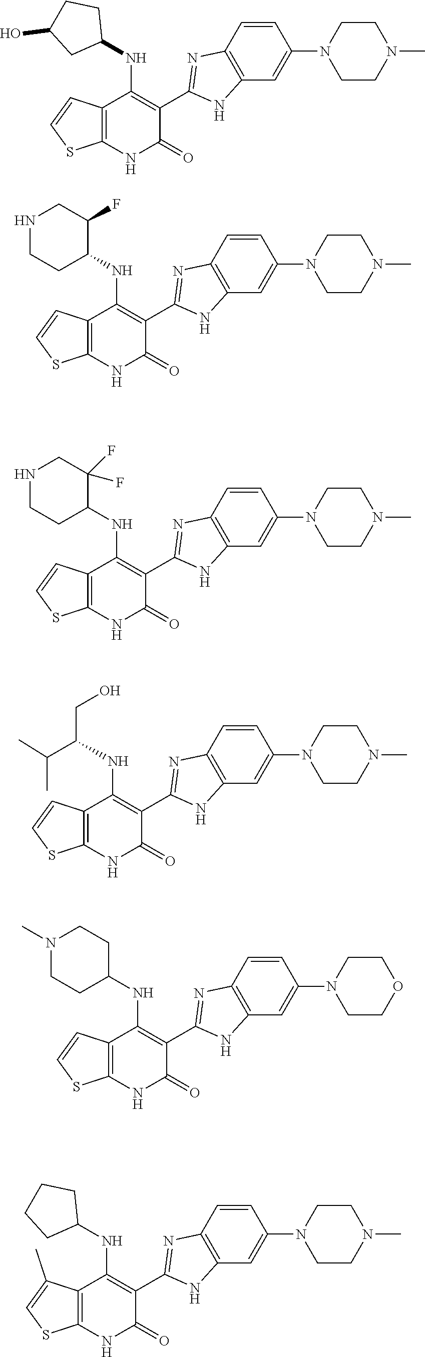

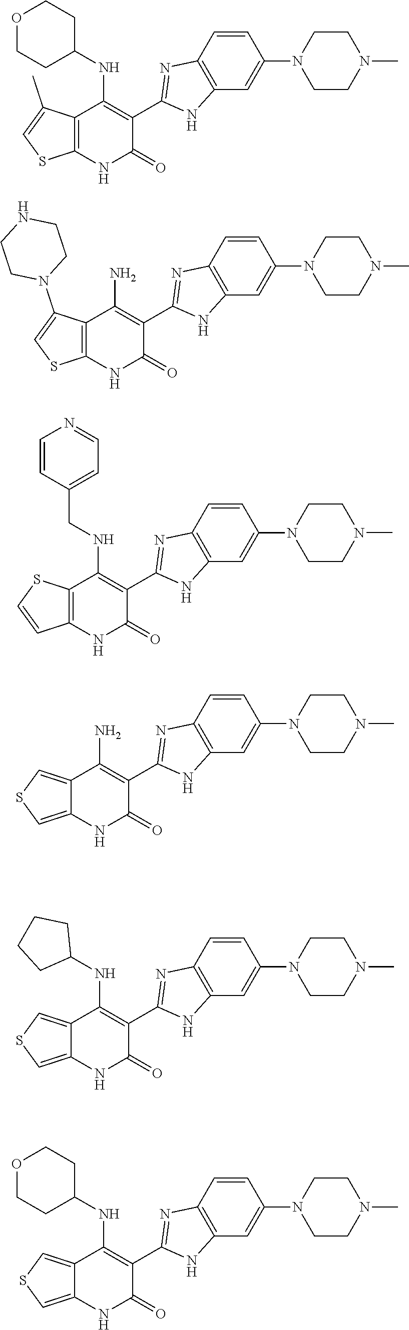 [SMILES: Cc1csc2[nH]c(=O)c(-c3nc4ccc(N5CCN(C)CC5)cc4[nH]3)c(NC3CCOCC3)c12 ; CN1CCN(c2ccc3nc(-c4c(N)c5c(N6CCNCC6)csc5[nH]c4=O)[nH]c3c2)CC1 ; CN1CCN(c2ccc3nc(-c4c(NCc5ccncc5)c5sccc5[nH]c4=O)[nH]c3c2)CC1 ; CN1CCN(c2ccc3nc(-c4c(N)c5cscc5[nH]c4=O)[nH]c3c2)CC1 ; CN1CCN(c2ccc3nc(-c4c(NC5CCCC5)c5cscc5[nH]c4=O)[nH]c3c2)CC1 ; CN1CCN(c2ccc3nc(-c4c(NC5CCOCC5)c5cscc5[nH]c4=O)[nH]c3c2)CC1]

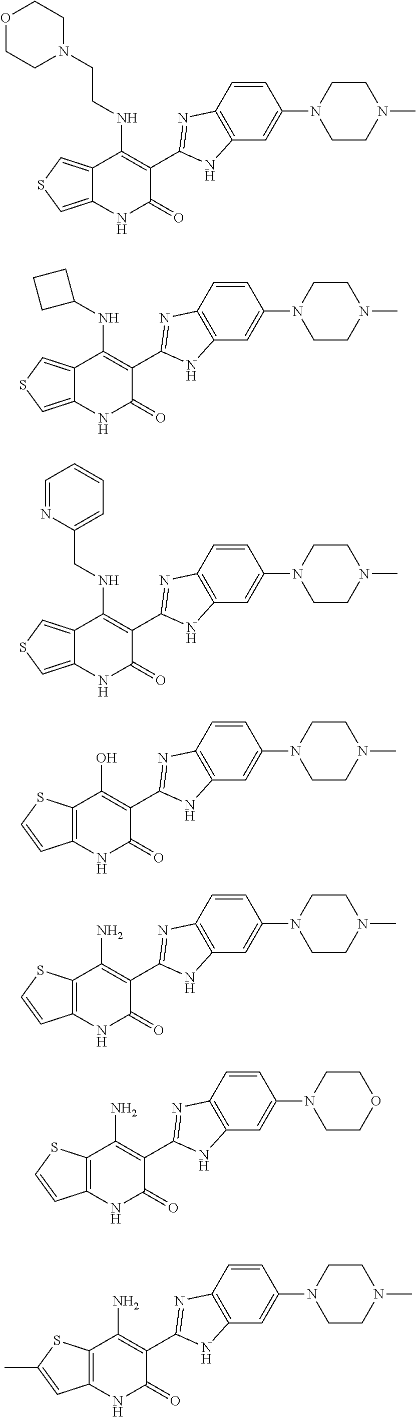

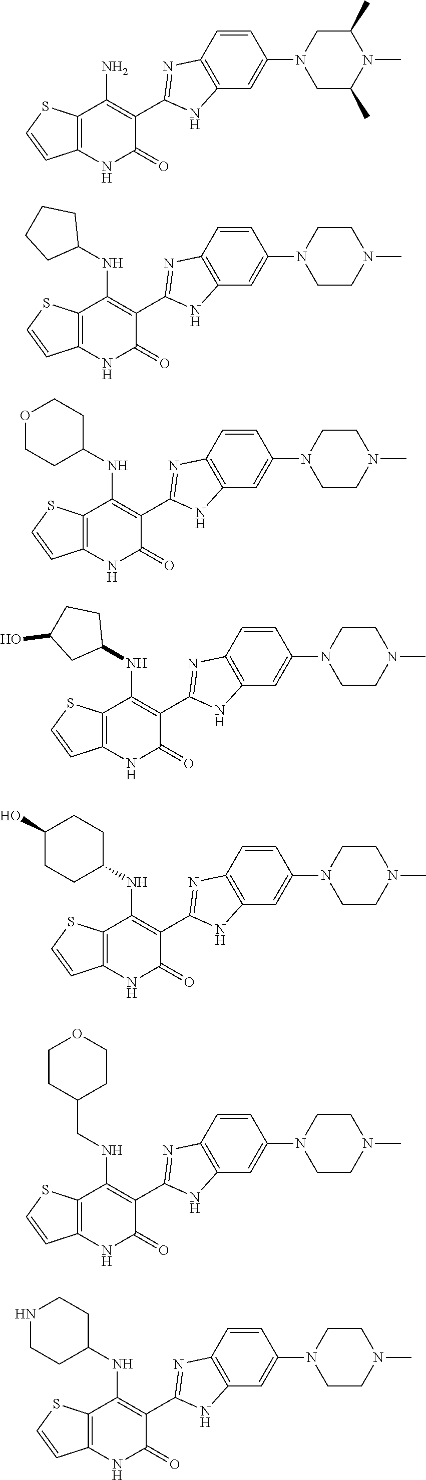

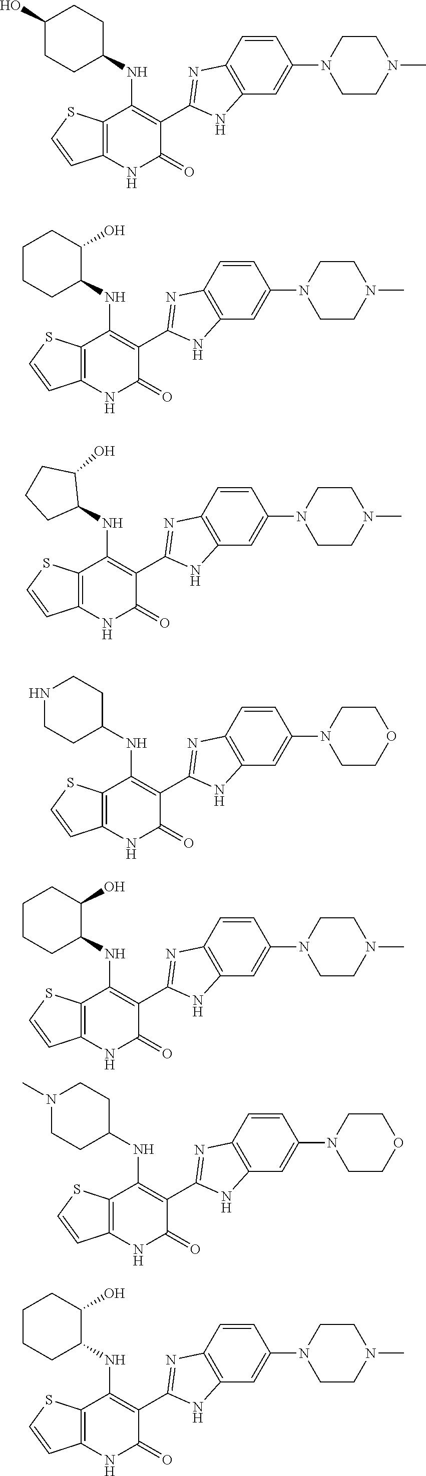

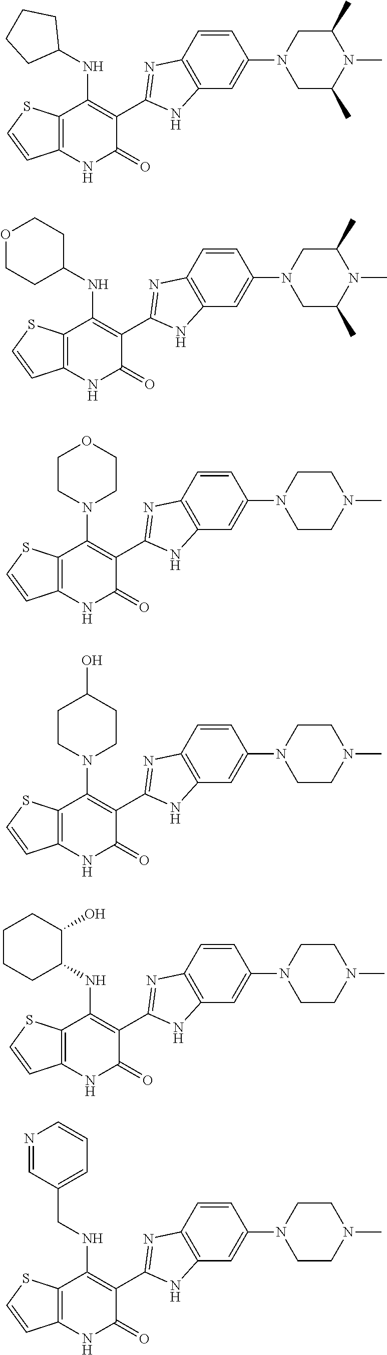 [SMILES: C[C@@H]1CN(c2ccc3nc(-c4c(NC5CCCC5)c5sccc5[nH]c4=O)[nH]c3c2)C[C@H](C)N1C ; C[C@@H]1CN(c2ccc3nc(-c4c(NC5CCOCC5)c5sccc5[nH]c4=O)[nH]c3c2)C[C@H](C)N1C ; CN1CCN(c2ccc3nc(-c4c(N5CCOCC5)c5sccc5[nH]c4=O)[nH]c3c2)CC1 ; CN1CCN(c2ccc3nc(-c4c(N5CCC(O)CC5)c5sccc5[nH]c4=O)[nH]c3c2)CC1 ; CN1CCN(c2ccc3nc(-c4c(N[C@@H]5CCCC[C@@H]5O)c5sccc5[nH]c4=O)[nH]c3c2)CC1 ; CN1CCN(c2ccc3nc(-c4c(NCc5cccnc5)c5sccc5[nH]c4=O)[nH]c3c2)CC1]

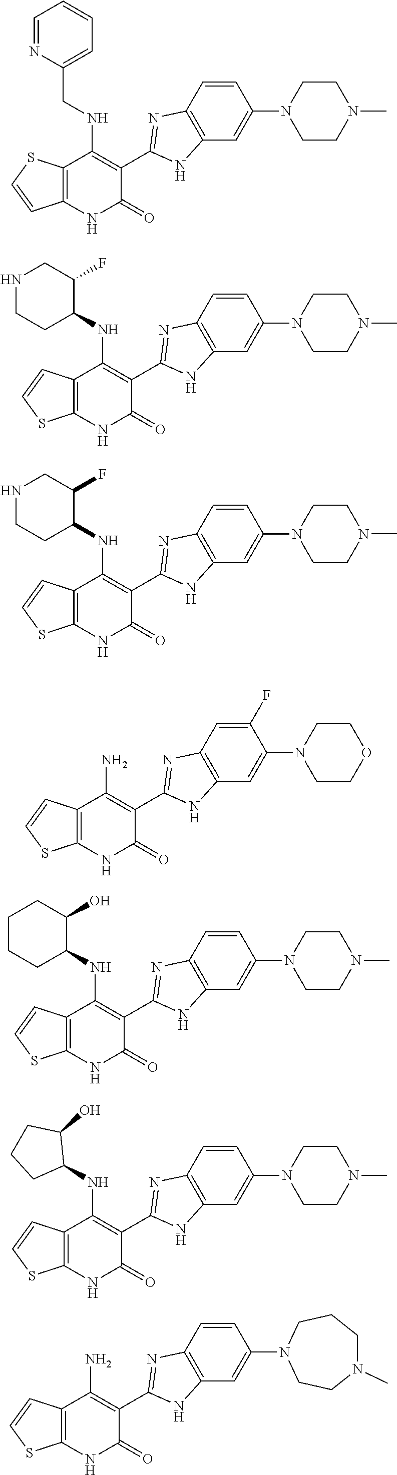

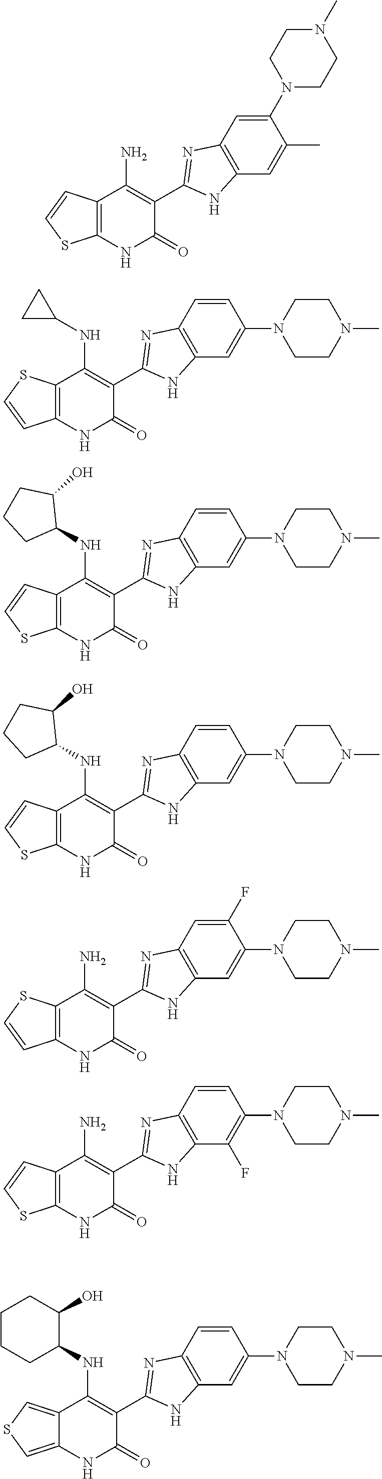

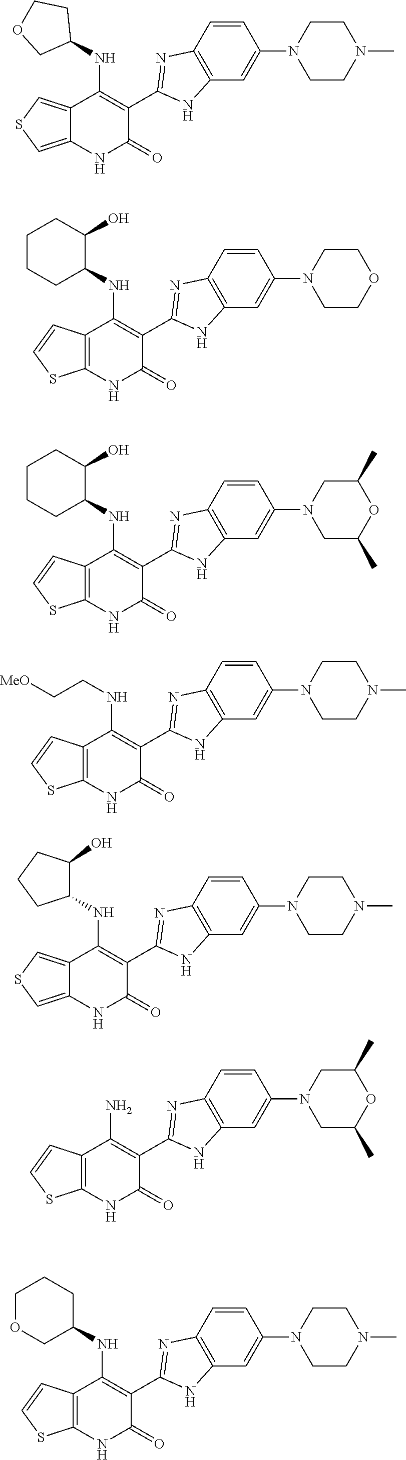

“Pharmaceutically acceptable salt” refers to a non-toxic salt form of a compound of this disclosure. Pharmaceutically acceptable salts of the compounds used in the disclosed methods include those derived from suitable inorganic and organic acids. Pharmaceutically acceptable salts are well known in the art. Suitable pharmaceutically acceptable salts are, e.g., those disclosed in Berge, S. M., et al. J. Pharma. Sci. 66:1-19 (1977). Non-limiting examples of pharmaceutically acceptable salts disclosed in that article include: acetate; benzenesulfonate; benzoate; bicarbonate; bitartrate; bromide; calcium edetate; camsylate; carbonate; chloride; citrate; dihydrochloride; edetate; edisylate; estolate; esylate; fumarate; gluceptate; gluconate; glutamate; glycollylarsanilate; hexylresorcinate; hydrabamine; hydrobromide; hydrochloride; hydroxynaphthoate; iodide; isethionate; lactate; lactobionate; malate; maleate; mandelate; mesylate; methylbromide; methylnitrate; methylsulfate; mucate; napsylate; nitrate; pamoate (embonate); pantothenate; phosphate/diphosphate; polygalacturonate; salicylate; stearate; subacetate; succinate; sulfate; tannate; tartrate; teociate; triethiodide; benzathine; chloroprocaine; choline; diethanolamine; ethylenediamine; meglumine; procaine; aluminum; calcium; lithium; magnesium; potassium; sodium; and zinc.

Non-limiting examples of pharmaceutically acceptable salts derived from appropriate acids include: salts formed with inorganic acids, such as hydrochloric acid, hydrobromic acid, phosphoric acid, sulfuric acid, or perchloric acid; salts formed with organic acids, such as acetic acid, oxalic acid, maleic acid, tartaric acid, citric acid, succinic acid, or malonic acid; and salts formed by using other methods used in the art, such as ion exchange. Additional non-limiting examples of pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. In one embodiment, the compound used in the disclosed methods is a mono HCl salt of Compound 1. In one embodiment, the compound used in the disclosed methods is a di-HCl salt of Compound 1. In one embodiment, the compound used in the disclosed methods is a 1:1 tartrate salt of Compound 1, wherein the molar ratio between the Compound 1 and tartaric acid is 1:1. In one embodiment, the compound used in the disclosed methods is a 1:1 maleate salt of Compound 1. In one embodiment, the compound used in the disclosed methods is a 1:1 mesylate salt of Compound 1. In one embodiment, the compound used in the disclosed methods is a 1:1 tartrate salt of Compound 1, wherein the molar ratio between the Compound 1 and tartaric acid is 1:1 and the salt is in the form of a polymorph characterized by XRPD peaks at 11.9°, 15.4°, 16.9°, and 17.2°±0.2 in 20. The polymorph can be prepared by crystallization of Compound 1 in a mixture of an aqueous acetic acid solution and an aqueous solution of L-(+)-tartaric acid, which is disclosed in U.S. Provisional Application Ser. No. 63/022,867, filed May 11, 2020, the entire teachings of which are incorporated herein by reference.

The term “alkyl” used alone or as part of a larger moiety, such as “alkoxy” or “haloalkyl” and the like, means saturated aliphatic straight-chain or branched monovalent hydrocarbon radical. Unless otherwise specified, an alkyl group typically has 1-6 carbon atoms, i.e. (C₁-C₆)alkyl. As used herein, a “(C₁-C₆)alkyl” group means a radical having from 1 to 6 carbon atoms in a linear or branched arrangement. Examples include methyl, ethyl, n-propyl, iso-propyl etc.

“Alkylene” refers to a bivalent straight or branched alkyl group typically with 1-6 carbon atoms, e.g., —(CH₂)ₙ—, wherein n is an integer from 1 to 6.

“Alkoxy” means an alkyl radical attached through an oxygen linking atom, represented by —O-alkyl. For example, “(C₁-C₄)alkoxy” includes methoxy, ethoxy, propoxy, and butoxy.

The terms “haloalkyl” and “haloalkoxy” means alkyl or alkoxy, as the case may be, substituted with one or more halogen atoms. The term “halogen” means F, C₁, Br or I. Preferably the halogen in a haloalkyl or haloalkoxy is F.

“Alkenyl” means branched or straight-chain monovalent hydrocarbon radical containing at least one double bond. Alkenyl may be mono or polyunsaturated, and may exist in the E or Z configuration. Unless otherwise specified, an alkenyl group typically has 2-6 carbon atoms, i.e. (C₂-C₆)alkenyl. For example, “(C₂-C₆)alkenyl” means a radical having from 2-6 carbon atoms in a linear or branched arrangement.

“Alkynyl” means branched or straight-chain monovalent hydrocarbon radical containing at least one triple bond. Unless otherwise specified, an alkynyl group typically has 2-6 carbon atoms, i.e. (C₂-C₆)alkynyl. For example, “(C₂-C₆)alkynyl” means a radical having from 2-6 carbon atoms in a linear or branched arrangement.

“Cycloalkyl” means a saturated aliphatic cyclic hydrocarbon radical, typically containing from 3-8 ring carbon atoms, i.e., (C₃-C₈)cycloalkyl. (C₃-C₈)cycloalkyl includes, but is not limited to, cyclopropyl, cyclobutyl, cyclopentyl, cyclohexyl, cycloheptyl and cyclooctyl.

As used herein, the term “bridged” used alone or as part of a larger moiety as in “bridged cycloalkyl” or “bridged heterocyclyl” refers to a ring system which includes two rings that share at least three adjacent ring atoms. Bridged cycloalkyl typically contains 6-12 ring carbon atoms. Bridged heterocyclyl typically have 6-12 ring atoms selected from carbon and at least one (typically 1 to 4, more typically 1 or 2) heteroatom (e.g., oxygen, nitrogen or sulfur).

The term “aryl” used alone or as part of a larger moiety as in “arylalkyl”, “arylalkoxy”, or “aryloxyalkyl”, means a carbocyclic aromatic ring. It also includes a phenyl ring fused with a cycloalkyl group. The term “aryl” may be used interchangeably with the terms “aryl ring” “carbocyclic aromatic ring”, “aryl group” and “carbocyclic aromatic group”. An aryl group typically has six to fourteen ring atoms. Examples includes phenyl, naphthyl, anthracenyl, 1,2-dihydronaphthyl, 1,2,3,4-tetrahydronaphthyl, fluorenyl, indanyl, indenyl and the like. A “substituted aryl group” is substituted at any one or more substitutable ring atom, which is a ring carbon atom bonded to a hydrogen.

The term “heteroaryl”, “heteroaromatic”, “heteroaryl ring”, “heteroaryl group”, “heteroaromatic ring”, and “heteroaromatic group”, are used interchangeably herein. “Heteroaryl” when used alone or as part of a larger moiety as in “heteroarylalkyl” or “heteroarylalkoxy”, refers to aromatic ring groups having five to fourteen ring atoms selected from carbon and at least one (typically 1 to 4, more typically 1 or 2) heteroatoms (e.g., oxygen, nitrogen or sulfur). “Heteroaryl” includes monocyclic rings and polycyclic rings in which a monocyclic heteroaromatic ring is fused to one or more other aryl, heterocyclyl or heteroaromatic rings. As such, “5-14 membered heteroaryl” includes monocyclic, bicyclic or tricyclic ring systems.

Examples of monocyclic 5-6 membered heteroaryl groups include furanyl (e.g., 2-furanyl, 3-furanyl), imidazolyl (e.g., N-imidazolyl, 2-imidazolyl, 4-imidazolyl, 5-imidazolyl), isoxazolyl (e.g., 3-isoxazolyl, 4-isoxazolyl, 5-isoxazolyl), oxadiazolyl (e.g., 2-oxadiazolyl, 5-oxadiazolyl), oxazolyl (e.g., 2-oxazolyl, 4-oxazolyl, 5-oxazolyl), pyrazolyl (e.g., 3-pyrazolyl, 4-pyrazolyl), pyrrolyl (e.g., 1-pyrrolyl, 2-pyrrolyl, 3-pyrrolyl), pyridyl (e.g., 2-pyridyl, 3-pyridyl, 4-pyridyl), pyrimidinyl (e.g., 2-pyrimidinyl, 4-pyrimidinyl, 5-pyrimidinyl), pyridazinyl (e.g., 3-pyridazinyl), thiazolyl (e.g., 2-thiazolyl, 4-thiazolyl, 5-thiazolyl), isothiazolyl, triazolyl (e.g., 2-triazolyl, 5-triazolyl), tetrazolyl (e.g., tetrazolyl), and thienyl (e.g., 2-thienyl, 3-thienyl). Examples of polycyclic aromatic heteroaryl groups include carbazolyl, benzimidazolyl, benzothienyl, benzofuranyl, isobenzofuranyl, indolyl, benzotriazolyl, benzothiazolyl, benzoxazolyl, quinolinyl, isoquinolinyl, indazolyl, isoindolyl, acridinyl, or benzisoxazolyl. A “substituted heteroaryl group” is substituted at any one or more substitutable ring atom, which is a ring carbon or ring nitrogen atom bonded to a hydrogen.

“Heterocyclyl” means a saturated or unsaturated non-aromatic 3-12 membered ring radical optionally containing one or more double bonds. It can be monocyclic, bicyclic, tricyclic, or fused. The heterocycloalkyl contains 1 to 4 heteroatoms, which may be the same or different, selected from N, O or S. The heterocyclyl ring optionally contains one or more double bonds and/or is optionally fused with one or more aromatic rings (e.g., phenyl ring). The term “heterocyclyl” is intended to include all the possible isomeric forms. Examples of heterocycloalkyl include, but are not limited to, azetidinyl, morpholinyl, thiomorpholinyl, pyrrolidinonyl, pyrrolidinyl, piperidinyl, piperazinyl, hydantoinyl, valerolactamyl, oxiranyl, oxetanyl, dihydroimidazole, dihydrofuranyl, dihydropyranyl, dihydropyridinyl, dihydropyrimidinyl, dihydrothienyl, dihydrothiophenyl, dihydrothiopyranyl, tetrahydroimidazole, tetrahydrofuranyl, tetrahydropyranyl, tetrahydrothienyl, tetrahydropyridinyl, tetrahydropyrimidinyl, tetrahydrothiophenyl, and tetrahydrothiopyranyl. Examples of polycyclic heterocycloalkyl groups include dihydroindolyl, dihydroisoindolyl, dihydrobenzimidazolyl, dihydrobenzothienyl, dihydrobenzofuranyl, dihydroisobenzofuranyl, dihydrobenzotriazolyl, dihydrobenzothiazolyl, dihydrobenzoxazolyl, dihydroquinolinyl, tetrahydroquinolinyl, dihydroisoquinolinyl, tetrahydroisoquinolinyl, dihydroindazolyl, dihydroacridinyl, tetrahydroacridinyl, dihydrobenzisoxazolyl, chroman, chromene, isochroman and isochromene.

A “subject” is a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

“Treat,” “treating,” or “treatment,” when used in connection with a subject with aberrant cytokine release from disease or condition, includes improving the effects or symptoms of aberrant cytokine release from the disease or disease or condition, e.g., lessening, reducing, modulating, ameliorating, and/or eliminating the effects of aberrant cytokine release. “Treat,” “treating,” or “treatment,” when used in connection with a subject at risk of aberrant cytokine release from disease or condition, includes reducing the severity of symptoms of aberrant cytokine release, when they develop, or to delay the onset of the symptoms. A subject “at risk” of developing aberrant cytokine release has a disease or condition known to develop aberrant cytokine release (or cytokine release syndrome or cytokine storm) in a subgroup of subjects. Treatment is preferably before the onset of aberrant cytokine release. Improvements in or lessening the severity of any symptom of the disorder or condition can be readily assessed according to standard methods and techniques known in the art.

“Effective amount” means an amount when administered to the subject which results in beneficial or desired results, including lessening, that results in the improvement the effects or symptoms of aberrant cytokine release from the disease or disease or condition. When administered to a subject “at risk” of developing aberrant cytokine release, “effective amount” means an amount which results in beneficial or desired results, including reducing the severity of symptoms of aberrant cytokine release, when they develop, or to delay the onset of the symptoms.

The precise amount of compound administered to provide an “effective amount” to the subject will depend on the mode of administration, the type, and severity of the disease or condition, and on the characteristics of the subject, such as general health, age, sex, body weight, and tolerance to drugs. The skilled artisan will be able to determine appropriate dosages depending on these and other factors. Suitable dosages are known for approved therapeutic agents and can be adjusted by the skilled artisan according to the condition of the subject, the type of condition(s) being treated and the amount of a compound of the invention being used by following, for example, dosages reported in the literature and recommended in the Physician's Desk Reference (57th ed., 2003). For example, an effective amount can be given in unit dosage form (e.g., 0.1 mg to about 50 g per day, alternatively from 1 mg to about 5 grams per day; and in another alternatively from 10 mg to 1 gram per day).

The compounds used in the disclosed methods can be administered to a patient in a variety of forms depending on the selected route of administration, as will be understood by those skilled in the art. The compounds of the present teachings may be administered, for example, by oral, parenteral, buccal, sublingual, nasal, rectal, patch, pump or transdermal administration and the pharmaceutical compositions formulated accordingly. Parenteral administration includes intravenous, intraperitoneal, subcutaneous, intramuscular, transepithelial, nasal, intrapulmonary, intrathecal, rectal and topical modes of administration. Parenteral administration can be by continuous infusion over a selected period of time.

The compounds used in the disclosed methods can be suitably formulated into pharmaceutical compositions for administration to a subject. These pharmaceutical compositions optionally include one or more pharmaceutically acceptable carriers and/or diluents therefor, such as lactose, starch, cellulose and dextrose. Other excipients, such as flavoring agents; sweeteners; and preservatives, such as methyl, ethyl, propyl and butyl parabens, can also be included. More complete listings of suitable excipients can be found in the Handbook of Pharmaceutical Excipients (5^{th} Ed., Pharmaceutical Press (2005)). A person skilled in the art would know how to prepare formulations suitable for various types of administration routes. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999. The carriers, diluents and/or excipients are “acceptable” in the sense of being compatible with the other ingredients of the pharmaceutical composition and not deleterious to the recipient thereof.

Typically, for oral therapeutic administration, a compound used in the disclosed methods may be incorporated with excipient and used in the form of ingestible tablets, buccal tablets, troches, capsules, elixirs, suspensions, syrups, wafers, and the like.

Typically for parenteral administration, solutions of a compound used in the disclosed methods can generally be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms.

Typically, for injectable use, sterile aqueous solutions or dispersion of, and sterile powders of, a compound described herein for the extemporaneous preparation of sterile injectable solutions or dispersions are appropriate.

For nasal administration, the compounds used in the disclosed methods can be formulated as aerosols, drops, gels and powders. Aerosol formulations typically comprise a solution or fine suspension of the active substance in a physiologically acceptable aqueous or non-aqueous solvent and are usually presented in single or multi-dose quantities in sterile form in a sealed container, which can take the form of a cartridge or refill for use with an atomizing device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. The aerosol dosage forms can also take the form of a pump-atomizer.

For buccal or sublingual administration, the compounds used in the disclosed methods can be formulated with a carrier such as sugar, acacia, tragacanth, or gelatin and glycerine, as tablets, lozenges or pastilles.

For rectal administration, the compounds used in the disclosed methods can be formulated in the form of suppositories containing a conventional suppository base such as cocoa butter.

### EXAMPLES

The following examples are intended to be illustrative and are not meant in any way to limit the scope of the disclosure.

The following compounds were used in Examples 1-6:

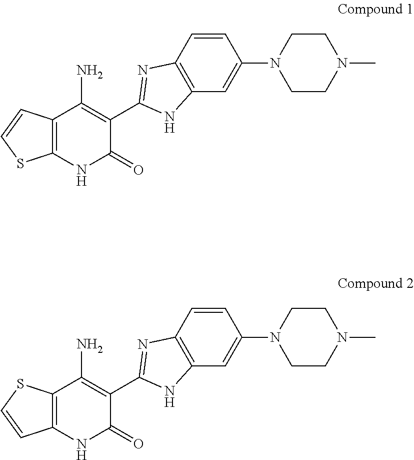 [SMILES: CN1CCN(c2ccc3nc(-c4c(N)c5ccsc5[nH]c4=O)[nH]c3c2)CC1 ; CN1CCN(c2ccc3nc(-c4c(N)c5sccc5[nH]c4=O)[nH]c3c2)CC1]

### Example 1. Compound 1 Suppresses Human Immune Cell Activation and Proliferation

Peripheral blood from healthy human donors was obtained from the Hematology Malignancy Tissue Bank at the University Health Network. PBMCs were prepared from the blood by density gradient centrifugation using Ficoll-Paque PLUS by manufacturer's instructions (GE Healthcare Life Sciences). PBMCs were frozen at 20×10⁶ cells/vial in 90% heat-inactivated fetal bovine serum (FBS) and 10% dimethylsulfoxide (DMSO) and stored in liquid nitrogen until use. PBMCs (2×10⁵ cells) were treated with either Compound 1 or DMSO, and anti-CD3 (clone OKT3, 1 ng/ml) and anti-CD28 (clone CD28.2, 100 ng/ml) antibodies, phytohemagglutinin (PHA) (3 μg/ml) or the superantigen staphylococcal enterotoxin B (SEB) (1 μg/ml) in RPMI 1640 medium containing 10% heat-inactivated FBS, 2-mercaptoethanol and penicillin-streptomycin antibiotics at 37° C., 5% CO₂, and 100% humidity. After 24 or 48 hours, the cells were stained with antibodies specific for the indicated cell subsets and activation markers for measurement by flow cytometry. The results are shown in FIG. 1A: Top, plots depict CD25, CD69 and CD62L expression by gated CD3⁺CD4⁺ T-cells. Bottom, plots depict CD25, CD69 and CD62L expression by gated CD3⁺CD8⁺ T-cells. Following activation, T-cells regulate the cell surface expression of activation markers, rapidly proliferate and acquire effector functions. Compound 1 treatment of PBMCs resulted in a titratable inhibition of CD4⁺ and CD8⁺ T-cell activation by anti-CD3 and anti-CD28 antibodies, PHA or SEB as shown by reduced cell surface expression of CD25 (IL-2 receptor alpha chain) and CD69 (type II C-lectin receptor), and reduced shedding of CD62L (L-selectin). Data are representative of several independent experiments utilizing different PBMC samples, and are reported as the mean fluorescence intensity (MFI)±standard deviation (SD) of duplicate wells.

PBMCs (2×10⁵ cells) were treated with either Compound 1 or DMSO, and anti-CD3 and anti-CD28 antibodies, PHA or SEB in RPMI 1640 medium containing 10% heat-inactivated FBS, 2-mercaptoethanol and penicillin-streptomycin antibiotics at 37° C., 5% CO₂, and 100% humidity. After 24 hours, the cells were labeled with ³H-thymidine for an additional 18 hours to measure lymphocyte proliferation by liquid scintillation counting. Proliferation of anti-CD3 antibody and anti-CD28 antibody-, PHA- or SEB-activated lymphocytes was inhibited by Compound 1 treatment, as shown in FIG. 1B. Similar data were obtained at 48 hours (data not shown). Data are representative of several independent experiments utilizing different PBMC samples, and are reported as the mean counts per minute (CPM)±standard deviation (SD) of duplicate wells.

### Example 2. Compound 1 Inhibits Lymphocyte Proliferation in an Allogenic Mixed Lymphocyte Reaction (MLR)

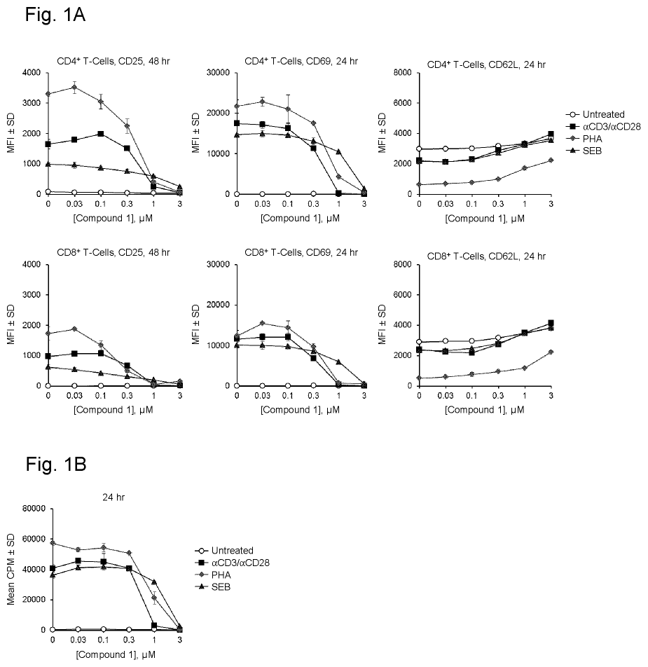

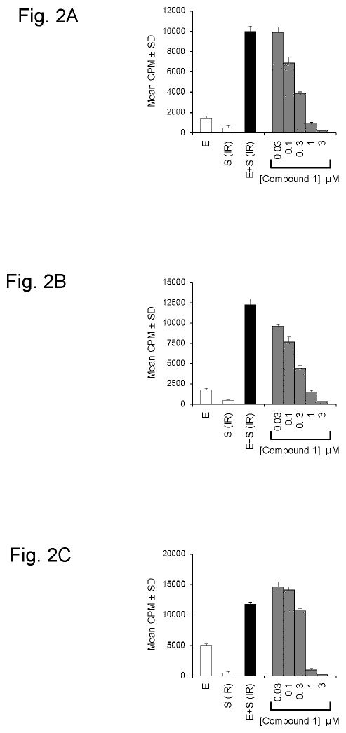

The allogenic MLR is a cell proliferation assay where one population of lymphocytes (effector cells) is stimulated to proliferate by another genetically distinct population of lymphocytes (stimulator cells), which have been rendered non-proliferative. PBMCs [2×10⁵ cells, effector (E) population] and irradiated (IR) allogenic PBMCs [1×10⁵ cells, stimulator (S) population] were treated with either Compound 1 or DMSO in RPMI 1640 medium containing 10% heat-inactivated FBS, 2-mercaptoethanol and penicillin-streptomycin antibiotics at 37° C., 5% CO₂, and 100% humidity. After 4 days, the cells were labeled with ³H-thymidine for an additional 18 hours to measure lymphocyte proliferation by liquid scintillation counting. Mixing of the two PBMC samples stimulated lymphocyte proliferation, whereas Compound 1 treatment resulted in a dose-dependent inhibition of lymphocyte proliferation. Multiple independent experiments utilizing different effector/stimulator pairs were conducted. See FIG. 2A-2C. Data are reported as the mean counts per minute (CPM)±standard deviation (SD) of triplicate wells.

### Example 3. Compound 1 Suppresses Effector Cytokine Secretion

PBMCs (2×10⁵ cells) were treated with either Compound 1 or DMSO, and anti-CD3 and anti-CD28 antibodies in RPMI 1640 medium containing 10% heat-inactivated FBS, 2-mercaptoethanol and penicillin-streptomycin antibiotics at 37° C., 5% CO₂, and 100% humidity. After 24 hours, cytokine levels in culture supernatants were determined by a LEGENDplex Human Th Cytokine Panel by manufacturer's instructions (BioLegend, Inc.).

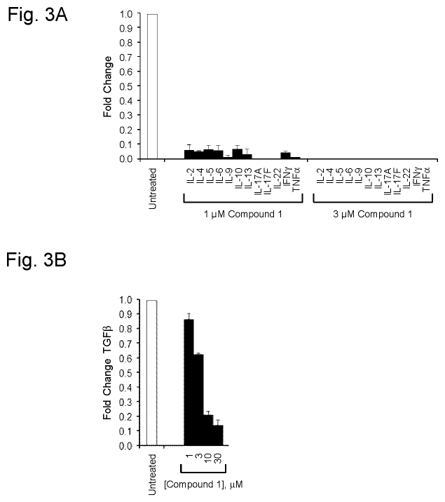

In the presence of Compound 1, the level of all measured cytokines decreased, including IL-2, IL-6, IFNγ and TNFα. See FIG. 3A. Data are representative of several independent experiments utilizing different human PBMC samples, and are reported as the fold change for Compound 1 relative to the DMSO control of duplicate wells.

Cancer-associated fibroblasts (CAFs) are used as a model system to investigate TGFβ cytokine production. C₅₇BL/6 mice were obtained from The Jackson Laboratory. The Institutional Animal Care and Use Committee of the University Health Network approved all animal procedures. CAFs were obtained from C₅₇BL/6 mice by growing MC38-CEA mouse colon cancer xenografts subcutaneously in a conventional manner. When tumors reached a size of approximately 1000 mm³ they were excised and disaggregated, and CAFs were isolated with a Tumor-Associated Fibroblast Isolation Kit (Miltenyi Biotec). Isolated CAFs were grown with a MesenCult Expansion Kit (STEMCELL Technologies, Inc.) at 37° C., 5% CO₂, 3% O₂, and 100% relative humidity. CAFs were seeded into a 96-well plate in DMEM medium containing 10% FBS 24 hours before treatment with either Compound 1 or DMSO. After 3 days, latent TGFβ was determined by a Mouse Latent TGFβ Legend Max kit by manufacturer's instructions (BioLegend, Inc.). Compound 1 inhibited TGFβ production by mouse primary CAFs. See FIG. 3B. Data are representative of several independent experiments, and are reported as the fold change for Compound 1 relative to the DMSO control of duplicate wells.

### Example 4. Compound 1 Effects on T-Cell and Monocyte Viability

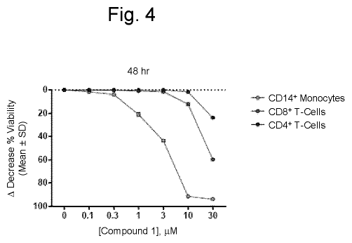

CD3⁺ T-cells and CD14⁺ monocytes were purified from PBMCs using Human CD3 MicroBeads and Human CD14 MicroBeads, respectively (Miltenyi Biotech). Purified CD3⁺ T-cells (2×10⁵ cells) or CD14⁺ monocytes (2×10⁵ cells) were treated with either Compound 1 or DMSO in RPMI 1640 medium containing 10% heat-inactivated FBS, 2-mercaptoethanol and penicillin-streptomycin antibiotics at 37° C., 5% CO₂, and 100% humidity. After 48 hours, the cells were stained with antibodies specific for the indicated cell subsets, and annexin V and 7-aminoactinomycin D (7-AAD) cell viability dyes for measurement by flow cytometry. Compound 1 treatment had no significant effect on the viability of resting CD4⁺ and CD8⁺ T-cells except at high concentrations (30 μM). Compound 1 treatment led to a dose-dependent loss of viability in resting CD14⁺ monocytes. See FIG. 4. Similar data were obtained at 24 and 72 hours (data not shown). Data are representative of several independent experiments utilizing different human PBMC samples, and are reported as the A decrease in percent viability of duplicate wells [mean±standard deviation (SD)].

### Example 5. Compound 2 Blocks Experimental Autoimmune Encephalomyelitis (EAE) Disease Progression

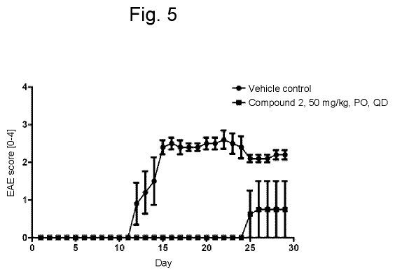

EAE is an animal model of multiple sclerosis (MS). In EAE, cytokines are critically involved in the autoantigen directed immune response, and in generating inflammation within the central nervous system. C₅₇BL/6 mice were obtained from The Jackson Laboratory. The Institutional Animal Care and Use Committee of the University Health Network approved all animal procedures. Mice were subcutaneously immunized with MOG35-55 peptide emulsified in Complete Freund's Adjuvant (CFA) supplemented with Mycobacterium tuberculosis. On days 0 and 2 after immunization, the mice were intraperitoneal injected with pertussis toxin. Clinical signs of EAE were monitored daily, according to the following criteria: 0, no disease; 1, decreased tail tone; 2, hind limb weakness or partial paralysis; 3, complete hind limb paralysis; 4, front and hind limb paralysis; 5, moribund state. During EAE induction, mice were given Compound 2 orally (PO) 50 mg/kg (n=4) or water (vehicle control; n=5) every day (QD). It is shown that Compound 2 blocks EAE disease progression. See FIG. 5. Data are reported as the mean score±standard error of the mean (SEM).

### Example 6. Compound 1 Does Not Cause Cytokine Production in Unstimulated Whole Blood

The potential for cytokine release syndrome in patients treated with Compound 1 was evaluated using a whole blood cytokine release assay (CRA). Fresh whole blood from a healthy human donor was diluted 4:1 with RPMI 1640 medium and cultured for 4 hours in the presence of Compound 1 or DMSO. Lipopolysaccharide (LPS) (1 μg/mL) was used as a positive control. Cytokine levels in serum samples were determined by a LEGENDplex Human Th Cytokine Panel by manufacturer's instructions (BioLegend, Inc.). Compound 1 did not induce levels of cytokines that would be predictive of cytokine release syndrome in vivo. Data listed in Table 1 below are representative of several independent experiments, and are reported as the mean fold change for Compound 1 relative to the DMSO control of duplicate wells.

The data listed in Table 1 show that Compound 1 does not cause release of cytokines from unstimulated whole blood.

TABLE 1

| | Sample | Compound 1 | | |
|---|---|---|---|---|
| Description | Source | Concentration | Endpoint Measured | Main Findings |
| Whole blood | Human | 0, 0.1, 0.3, 3, μM | Undiluted fresh whole | After treatment at all dose |
| cytokine | whole blood | | blood from a healthy | levels, the median concentration |
| release assay | | | donor was incubated | for all cytokines evaluated |
| (CRA) | | | with Compound 1 for | (IL-2, IL-4, IL-5, IL-6, |
| | | | 4 hours at 37° C. | IL-9, IL-10, IL-13, IL-17A, |
| | | | Cytokine levels in | IL-17F, IL-21, IL-22, IFNγ |
| | | | plasma were measured by | and TNFα) was below the |
| | | | bead-based immunoassay. | sensitivity of the assay |
| | | | | as reported by the manufacturer |
| | | | | (sensitivity range = 1.1-3.2 |
| | | | | pg/ml). |

1. A method of treating a subject with aberrant cytokine release from a disease or condition or at risk of developing aberrant cytokine release from a disease or condition, comprising administering to the subject an effective amount of a compound represented by the following structural formula: 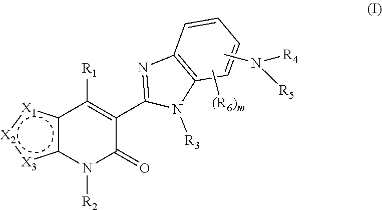
or a pharmaceutically acceptable salt thereof, wherein:
one of X₁, X₂, and X₃ is S, the other two are each independently CR;
R is H, —F, —Cl, —Br, —OH, —(C₁-C₄)alkyl, —(C₁-C₄)haloalkyl, —(C₁-C₄)alkoxy, —(C₁-C₄)alkylene-OH or 4-7 membered monocyclic heterocyclyl optionally substituted with 1-3 groups selected from —F, —Cl, —Br, —OH, —(C₁-C₄)alkyl, —(C₁-C₄)haloalkyl, —(C₁-C₄)alkoxy, or —CO₂—(C₁-C₄)alkyl;
R₁ is —NR^{a}R^{b} or —OR^{a1};
R^{a} for each occurrence is independently —H, —(C₁-C₆)alkyl, —(CH₂)ₙ—(C₃-C₇)cycloalkyl, —(CH₂)ₙ-3-7 membered monocyclic heterocyclyl, —(CH₂)ₙ-bridged (C₆-C₁₂)cycloalkyl, optionally substituted —(CH₂)ₙ-5-10 membered heteroaryl; or —(CH₂)ₙ-6-12 membered bridged heterocyclyl, wherein —(C₁-C₆)alkyl, —(CH₂)ₙ—(C₃-C₇)cycloalkyl, —(CH₂)ₙ-3-7 membered monocyclic heterocyclyl, —(CH₂)ₙ-bridged (C₆-C₁₂)cycloalkyl, —(CH₂)ₙ-5-10 membered heteroaryl, or —(CH₂)ₙ-6-12 membered bridged heterocyclyl, is optionally substituted with 1-3 groups selected from —F, —Cl, —Br, —CN, —NH₂, —OH, oxo, —(C₁-C₄)alkyl, —(C₁-C₄)haloalkyl, —(C₁-C₄)alkoxy, —(C₁-C₄)haloalkoxy, —(C₁-C₄)alkylene-OH, or —(C₁-C₄)alkylene-NH₂;
R^{b} for each occurrence is independently —H or —(C₁-C₆)alkyl; or,
R^{a} and R^{b}, together with the nitrogen to which they are attached, form —(C₃-C₁₀)heterocyclyl;
R^{a1} for each occurrence is independently —H, (C₁-C₆)alkyl, (C₃-C₁₀)cycloalkyl, 3-10 membered heterocyclyl, (C₆-C₁₀)aryl, or 3-10 membered heteroaryl;
R₂ and R₃ are independently H or —(C₁-C₄)alkyl;
R₄ and R₅, together with the nitrogen to which they are attached, form 4-7 membered monocyclic heterocyclyl or 6-12 membered bridged heterocyclyl, wherein the 4-7 membered monocyclic heterocyclyl or 6-12 membered bridged heterocyclyl is optionally substituted with 1-3 groups selected from —F, —Cl, —Br, —CN, —NH₂, —OH, oxo, —(C₁-C₄)alkyl, —(C₁-C₄)haloalkyl, —(C₁-C₄)alkoxy, —(C₁-C₄)haloalkoxy, —(C₁-C₄)alkylene-OH, or —(C₁-C₄)alkylene-NH₂;
R₆ for each occurrence is independently —F, —Cl, —Br, —CN, —NH₂, —OH, —(C₁-C₆)alkyl; —(C₁-C₆)haloalkyl, —(C₂-C₆)alkenyl, —(C₂-C₆)alkynyl, (C₃-C₆)cycloalkyl, —(C₁-C₆)alkoxy, —(C₁-C₆)haloalkoxy, —(C₁-C₆)alkylene-OH, or —(C₁-C₆)alkylene-NH₂;
m is 0, 1, 2, or 3; and
n is 0, 1, or 2.

2. The method of claim 1, wherein the subject has aberrant cytokine release from the disease or condition or the subject is at risk of developing aberrant cytokine release from the disease or condition.

3. (canceled)

4. The method of claim 2, wherein the aberrant cytokine release is cytokine release syndrome or cytokine storm syndrome.

5. The method of claim 1, wherein the subject has aberrant cytokine release or is at risk of developing aberrant cytokine release from activated T-cells, activated natural killer (NK) cells, activated dendritic cells, activated macrophages, activated B-cells, or antitumor cell therapy, or
wherein the subject has aberrant cytokine release or is at risk of developing aberrant cytokine release from adoptive cell therapy using tumor-infiltrating lymphocyte (TIL) therapy, engineered T cell receptor (TCR) therapy, chimeric antigen receptor (CAR) T cell therapy and therapies that incorporate other immune cells, or
wherein the subject has aberrant cytokine release or is at risk of developing aberrant cytokine release from a chimeric antigen receptor (CAR) T cell therapy, or
wherein the subject has aberrant cytokine release or is at risk of developing aberrant cytokine release from a therapy with an antibody, or
wherein the subject has aberrant cytokine release or is at risk of developing aberrant cytokine release from a therapy with a non-protein-based cancer drug, or
wherein the subject has aberrant cytokine release or is at risk of developing aberrant cytokine release from a haploidentical donor stem cell transplantation.

6. (canceled)

7. The method of claim 4, wherein the subject has aberrant cytokine release or is at risk of developing aberrant cytokine release from chimeric antigen receptor (CAR) T cell therapy, and the CAR T cell therapy is tisagenlecleucel or axicabtagene ciloleucel.

8. (canceled)

9. The method of claim 4, wherein the subject has aberrant cytokine release or is at risk of developing aberrant cytokine release from a therapy with an antibody, and the antibody is a monoclonal antibody, an antibody fragment, a Fc-fusion protein or a bispecific antibody.

10. The method of claim 9, wherein the antibody is a bispecific antibody selected from bispecific T cell engager or BiTE.

11. (canceled)

12. The method of claim 9, wherein the antibody is a monoclonal antibody selected from an anti-PD-L1 antibody, an anti-CTLA-4 antibody, an anti-PD-1 antibody, anti-CD3 antibody, anti-CD20 antibody, anti-CD28 antibody, anti-CD52 antibody and anti-thymocyte globulin (ATG).

13. The method of claim 9, wherein the antibody is a monoclonal antibody selected from Nivolumab, Muromonab, Rituximab, Brentuximab, Theralizumab, Alemtuzumab, Obinutuzumab, Dacetuzumab, Pembrolizumab, Cemiplimab, Atezolizumab, Avelumab, Durvalumab and Ipilimumab; or the antibody is a bispecific T cell engager selected from Blinatumomab (Blincyto).

14. The method of claim 4, wherein the subject has aberrant cytokine release or is at risk of developing aberrant cytokine release from therapy with a non-protein-based cancer drug selected from oxaliplatin and lenalidomide.

15. The method of claim 1, wherein the disease or condition is an infectious disease; or
wherein the disease or condition is an auto-inflammatory disease or an autoimmune disease, or
wherein the disease or condition is selected from hemophagocytic lymphohistiocytosis (HLH), familial (primary) hemophagocytic lymphohistiocytosis (FHL), sporadic HLH, macrophage activation syndrome (MAS), chronic arthritis, systemic Juvenile Idiopathic Arthritis (sJIA), Still's Disease, a Cryopyrin-associated Periodic Syndrome (CAPS), Familial Cold Auto-inflammatory Syndrome (FCAS), Familial Cold Urticaria (FCU), Muckle-Well Syndrome (MWS), Chronic Infantile Neurological Cutaneous and Articular (CINCA) Syndrome, a cryopyrinopathy comprising inherited or de novo gain of function mutations in the NLRP3 gene, a hereditary auto-inflammatory disorder, acute pancreatitis, severe burn injury, acute radiation syndrome, trauma, acute respiratory distress syndrome, systemic inflammatory response syndrome, and tumor lysis syndrome, or
wherein the disease or condition is selected from cachexia, a chronic inflammatory response, sepsis, septic shock syndrome, traumatic brain injury, cerebral cytokine storm, graft versus host disease (GVHD), autoimmune diseases, multiple sclerosis, acute pancreatitis, or hepatitis, or
wherein the disease or condition is selected from myocarditis, Type 1 diabetes, Type 2 diabetes, thyroiditis, uveitis, encephalomyelitis, arthritis, lupus erythematosus, myositis, systemic sclerosis, Sjogren's syndrome and heart failure.

16. The method of claim 15, wherein the disease or condition is an infectious disease, and
wherein the infectious disease is viral, bacterial, fungal, helminthic, protozoan or hemorrhagic,
wherein the infectious disease is a viral infection selected from influenza, Arenaviridae, Filoviridae, Bunyaviridae, Flaviviridae, Rhabdoviridae and Cornaviridae, or
wherein the infectious disease is a viral infection selected from Epstein Barr virus, small pox, Ebola, Marburg, Crimean-Congo hemorrhagic fever (CCHF), South American hemorrhagic fever, dengue, yellow fever, Rift Valley fever, Omsk hemorrhagic fever virus, Kyasanur Forest, Junin, Machupo, Sabia, Guanarito, Garissa, Ilesha and Lassa, or
wherein the infectious disease is an influenza virus infection.

17-19. (canceled)

20. The method of claim 16, wherein the infectious disease is a viral infection by Coronaviridae which is from a virus selected from SARS, SARS-CoV-2, MERS, 229E, NL63, OC43, and HKUL.

21-25. (canceled)

26. The method of claim 15, wherein the disease or condition is an auto-inflammatory disease or an autoimmune disease, and the autoimmune disease or auto-inflammatory disease is selected from Type 1 diabetes, Type 2 diabetes, rheumatoid arthritis (RA), systemic lupus erythematosus (SLE), multiple sclerosis (MS), inflammatory bowel disease (Crohn's disease and ulcerative colitis), psoriasis, asthma, familial Mediterranean fever (FMF), Tumor Necrosis Factor (TNF) receptor-associated periodic syndrome (TRAPS), mevalonate kinase deficiency/hyperimmunoglobulin D syndrome (MKD/HIDS), Muckle-Wells syndrome (MWS), familial cold auto-inflammatory syndrome (FCAS), neonatal-onset multisystem inflammatory disease (NOMID), periodic fever, aphthous stomatitis, pharyngitis and adenitis (PFAPA syndrome), pyogenic sterile arthritis, pyoderma gangrenosum, acne (PAPA), deficiency of the interleukin-1 receptor antagonist (DIRA), Behcet's disease, Majeed Syndrome, Chronic recurrent multifocal osteomyelitis (CRMO), Schnitzler syndrome and Blau syndrome.

27-30. (canceled)

31. The method of claim 1, wherein the compound is represented by a structural formula selected from: 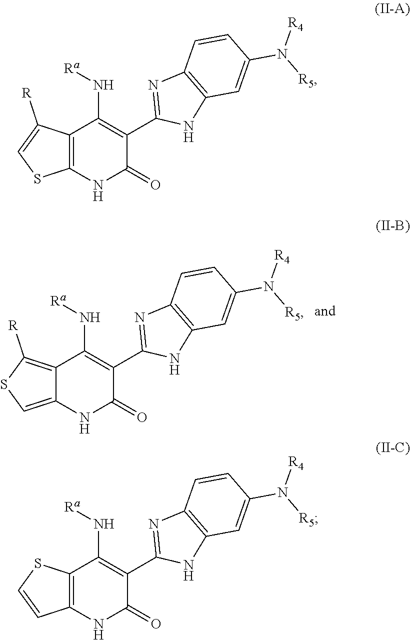
or a pharmaceutically acceptable salt thereof, wherein:
R is H, —(C₁-C₄)alkyl, —(C₁-C₄)alkoxy, N-piperazinyl optionally substituted with —CO₂—(C₁-C₄)alkyl;
R₄ and R₅, together with the nitrogen to which they are attached, form —N-alkyl-piperazinyl or morpholinyl, wherein the piperazinyl or morpholinyl is optionally substituted with 1-2 groups selected from —F, —Cl, —Br, —OH, —(C₁-C₄)alkyl, —(C₁-C₄)haloalkyl, or —(C₁-C₄)alkoxy;
R^{a} for each occurrence is independently —H, —(CH₂)ₙ—(C₃-C₆)cycloalkyl, —(CH₂)ₙ-3-6 membered monocyclic heterocyclyl, wherein the —(CH₂)ₙ—(C₃-C₆)cycloalkyl or —(CH₂)ₙ-3-6 membered monocyclic heterocyclyl is optionally substituted with 1-3 groups selected from —F, —Cl, —Br, —CN, —NH₂, —OH, —(C₁-C₄)alkyl, or —(C₁-C₄)alkoxy; and
n is 0 or 1.

32. (canceled)

33. The method of claim 31, wherein:
R is H;
R₄ and R₅, together with the nitrogen to which they are attached, form —N-methyl-piperazinyl or morpholinyl, both of which are optionally substituted with one or two methyl;
R^{a} for each occurrence is independently —H; —(C₃-C₆)cycloalkyl optionally substituted with —OH; —(CH₂)ₙ-tetrahydro-2H-pyran; morpholinyl; piperidinyl optionally substituted with —F, —OH or methyl; or tetrahydrofuranyl; and
n is 0 or 1.

34. The method of claim 15, wherein the compound is represented by the following structural formula: 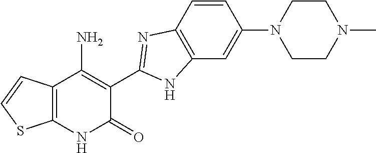 [SMILES: CN1CCN(c2ccc3nc(-c4c(N)c5ccsc5[nH]c4=O)[nH]c3c2)CC1]
or a pharmaceutically acceptable salt thereof.

35. The method of claim 15, wherein the compound is represented by the following structural formula: 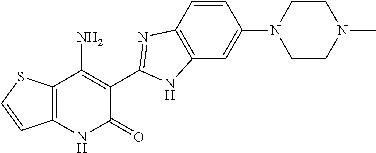 [SMILES: CN1CCN(c2ccc3nc(-c4c(N)c5sccc5[nH]c4=O)[nH]c3c2)CC1]
or a pharmaceutically acceptable salt thereof.

36. A method of treating a subject with a systemic inflammatory response from a disease or condition or a subject at risk of developing systemic inflammatory response from a disease or condition, comprising administering to the subject a compound from claim 1 or a pharmaceutically acceptable salt thereof.

37. The method of claim 36, wherein the disease or condition is an infectious disease, or
wherein the disease or condition is an auto-inflammatory disease or an autoimmune disease, or
wherein the disease or condition is selected from hemophagocytic lymphohistiocytosis (HLH), familial (primary) hemophagocytic lymphohistiocytosis (FHL), sporadic HLH, macrophage activation syndrome (MAS), chronic arthritis, systemic Juvenile Idiopathic Arthritis (sJIA), Still's Disease, a Cryopyrin-associated Periodic Syndrome (CAPS), Familial Cold Auto-inflammatory Syndrome (FCAS), Familial Cold Urticaria (FCU), Muckle-Well Syndrome (MWS), Chronic Infantile Neurological Cutaneous and Articular (CINCA) Syndrome, a cryopyrinopathy comprising inherited or de novo gain of function mutations in the NLRP3 gene, a hereditary auto-inflammatory disorder, acute pancreatitis, severe burn injury, acute radiation syndrome, trauma, acute respiratory distress syndrome, systemic inflammatory response syndrome, and tumor lysis syndrome, or
wherein the disease or condition is selected from cachexia, a chronic inflammatory response, sepsis, septic shock syndrome, traumatic brain injury, cerebral cytokine storm, graft versus host disease (GVHD), autoimmune diseases, multiple sclerosis, acute pancreatitis, or hepatitis, or
wherein the disease or condition is selected from myocarditis, Type 1 diabetes, Type 2 diabetes, thyroiditis, uveitis, encephalomyelitis, arthritis, lupus erythematosus, myositis, systemic sclerosis, Sjogren's syndrome and heart failure.
